SEQUENCE LISTING (3 sequences)

SEQ ID NO: 1 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 2 - protein, 552 aa, Homo sapiens
MGCSAKARWAAGALGVAGLLCAVLGAVMIVMVPSLIKQQVLKNVRIDPSSLSFNMWKEIPIPFYLSVYFFDVMNPSEILKGEKPQVRERGPYVYREFRHKSNITFNNNDTVSFLEYRTFQFQPSKSHGSESDYIVMPNILVLGAAVMMENKPMTLKLIMTLAFTTLGERAFMNRTVGEIMWGYKDPLVNLINKYFPGMFPFKDKFGLFAELNNSDSGLFTVFTGVQNISRIHLVDKWNGLSKVDFWHSDQCNMINGTSGQMWPPFMTPESSLEFYSPEACRSMKLMYKESGVFEGIPTYRFVAPKTLFANGSIYPPNEGFCPCLESGIQNVSTCRFSAPLFLSHPHFLNADPVLAEAVTGLHPNQEAHSLFLDIHPVTGIPMNCSVKLQLSLYMKSVAGIGQTGKIEPVVLPLLWFAESGAMEGETLHTFYTQLVLMPKVMHYAQYVLLALGCVLLLVPVICQIRSQVGAGQRAARADSHSLACWGKGASDRTLWPTAAWSPPPAAVLRLCRSGSGHCWGLRSTLASFACRVATTLPVLEGLGPSLGGGTGS

SEQ ID NO: 3 - protein, 7 aa, Homo sapiens
EVHHQKL

(19) United States
(12) Patent Application Publication
(10) Pub. No.: US 2015/0079111 A1
(43) Pub. Date: Mar. 19, 2015

(54) MECHANISM, DIAGNOSTIC, AND TREATMENTS FOR COMPLICATIONS OF RENAL FAILURE

(71) Applicant: Jonas AXELSSON, Stockholm (SE)

(72) Inventor: Jonas Axelsson, Stockholm (SE)

(21) Appl. No.: 14/397,219

(22) PCT Filed: Jun. 5, 2013
(86) PCT No.: PCT/IB2013/001658

§ 371 Date: Oct. 27, 2014

Related U.S. Application Data
(60) Provisional application No. 61/655,522, filed on Jun. 5, 2012.

(30) Foreign Application Priority Data
Jun. 5, 2013 (IB) PCT/IB2013/001658

(51) Int. Cl. A61K 31/4045; A61K 31/713; G01N 33/68; A61K 38/44; A61K 31/03; A61K 31/121; A61K 31/407; A61K 39/395

(52) U.S. Cl. CPC A61K 31/4045; A61K 31/407; A61K 31/713; A61K 39/3955; A61K 38/446; A61K 31/03; A61K 31/121; G01N 33/6893; C12Y 115/01001; G01N 2333/90209; G01N 2333/70596; G01N 2333/705; G01N 2800/347
USPC 424/172.1; 514/414; 514/410; 514/44 A; 424/94.4; 514/753; 514/689; 435/7.21; 435/7.4; 435/7.1; 435/7.92; 435/6.1

(57) ABSTRACT

Mechanisms for complications of renal failure and processes for treating the same are disclosed. They include contacting a biological fluid (e.g. blood) with a filter body, the filter body having immobilized thereon either a CD36 receptor or a CD36 receptor fragment or a SCARB1 receptor or a SCARB1 receptor fragment, the blood having a reduced quantity of oxidatively modified proteins after contacting the filter body. They also include administering an antagonist drug to a patient that blocks the binding activity of the RAGE receptor or the CD36 receptor or the SCARB1 receptor. Diagnostic methods are also provided.

This application claims priority to U.S. Provisional Patent Application Ser. No. 61/655,522, filed Jun. 5, 2012. The disclosure of that application is fully incorporated by reference herein.

### BACKGROUND

The present disclosure relates to kidney diseases and renal failure, processes for treating such, and devices to be used for treating such. It finds particular application in conjunction with particular cell-surface receptors which bind and internalize oxidatively modified proteins, and will be described with particular reference thereto. However, it is to be appreciated that the present disclosure is also amenable to other similar applications, such as diagnostics, etc.

It is well known that the risk of mortality from chronic kidney disease (CKD) is proportional to the loss of renal function. However, despite more than thirty years of advances in nephrological research, the reason behind the inverse relationship between mortality risk and renal function remains unknown. Well-designed studies have shown that increasing the dialysis dose using current technology is not able to effectively treat CKD. Furthermore, the normalization of hemoglobin using erythropoietin, normalization of serum phosphate, and lowering of lipids using statins have also yielded negative outcomes in studied CKD populations. In epidemiological studies, both oxidative stress and systemic inflammation have been linked to CKD morbidity and mortality. Pathology studies indicate that endothelial dysfunction, arterial calcification, and renin-angiotensin-aldosterone (RAS) overactivity characterize the majority of CKD patients, and also predict both progression of renal function decline and mortality.

Therefore, a viable treatment for kidney disease that elucidates and specifically targets the major determinants of morbidity and mortality is desired.

### BRIEF DESCRIPTION

Disclosed herein are processes and devices for treating the complications of acute or chronic renal failure, as well as the progression of the same.

Disclosed in embodiments herein are a process for treating renal failure and its complications, comprising contacting blood with a filter body, the filter body having immobilized thereon either a CD36 (accession number P16671) or SCARB1 (accession number Q8WTV0.1) receptor, an antibody against a CD36-binding motif or SCARB1-binding motif, or a CD36 or SCARB1 receptor fragment, the blood having a reduced quantity of oxidatively modified proteins after contacting the filter body.

The process may further comprise separating the biological fluid from the filter body. The filter body can be in the shape of a bead or a hollow fiber. The oxidatively modified proteins are generally advanced oxidation protein products or advanced glycation end products.

Also disclosed are separation mediums comprising a support and immobilized thereon either a CD36 or SCARB1 receptor, an antibody against a CD36-binding motif or SCARB1-binding motif, or a CD36 or SCARB1 receptor fragment.

Also disclosed herein are processes for treating complications of renal failure, comprising administering to a patient an antagonist drug that blocks the binding activity of the RAGE receptor.

The antagonist drug may include an anti-RAGE antibody or a small interfering RNA configured for silencing RAGE, or both the anti-RAGE antibody and the small interfering RNA. Sometimes, the antagonist drug is a glitazone, FPS-ZM1, or low anti-coagulant 2-O,3-O-desulfated heparin.

Also disclosed are processes for treating complications of renal failure, comprising administering to a patient an antagonist drug that blocks the binding activity of the CD36 receptor.

The antagonist drug can include an anti-CD36 antibody or a small interfering RNA configured for silencing CD36, or both the anti-CD36 antibody and the small interfering RNA. Sometimes, the antagonist drug is AP5055, AP5258, ursolic acid or a derivative thereof, or hexarelin.

Also described herein are processes for treating complications of renal failure, comprising administering to a patient an antagonist drug that blocks the binding activity of the SCARB1 receptor.

The antagonist drug can include an anti-SCARB1 antibody or a small interfering RNA configured for silencing SCARB1, or both the anti-SCARB1 antibody and the small interfering RNA. For example, the antagonist drug can be acetyl-salicylate, sodium-salicylate, ezetimibe, or enfuvirtide.

Also disclosed are processes for controlling the binding of oxidatively modified proteins to SCARB1, comprising administering to a patient a compound that binds to SCARB1. In specific embodiments, the compound can be a synthetic peptide or fusion protein, such as humanized E2 derived from the hepatitis C virus, a modified amino acid such as phosphatidylserine, or an antibody configured against one or more extracellular domains of SCARB1.

Also disclosed are processes for ameliorating the downstream effects of oxidatively modified proteins bound to CD36 or SCARB1 through the administration of a RAS signaling pathway modulator, an oxygen scavenger, an NADPH oxidase inhibitor, a pan-PKC inhibitor, or a PKCα inhibitor to a patient.

The RAS signaling pathway modulator can be enalapril, losartan, or spironolactone.

The oxygen scavenger may be copper-zinc superoxide dismutase, a pterin, a flavonoid, 2,3-dimethyl-6(2-dimethylaminoethyl)-6H-indolo-(2,3-b)quinoxaline, N-acetylcysteine, or ascorbic acid.

The NADPH oxidase inhibitor may be diphenyleneiodonium (DPI), apocynin, a procyanidin, Schisandrin B, or annexin peptide Ac2-26.

The pan-PKC inhibitor can be Gö6983, genistein, calphostin C, or GF109203X.

The PKCα inhibitor can be Gö6976, PKCα (C2-4) inhibitor peptide, aprinocarsen, or MT477.

Also described are processes for identifying patients at risk of tissue accumulation of aggregates containing oxidized proteins due to reduced renal clearance, comprising: measuring the concentration of at least one SCARB1 binding molecule in a bodily fluid of a patient. The SCARB1 binding molecule may be malondialdehyde (MDA) or pentosidine.

Also disclosed are processes for reducing tissue accumulation of aggregates containing oxidized proteins due to reduced renal clearance, for example vascular amyloid formation, comprising administering to a patient a compound that competitively binds to CD36 or SCARB1 or that modifies the expression or cell surface availability of these receptors. The compound can be (i) a small molecule such as hexarelin or sodium-salicylate, (ii) a synthetic peptide or fusion protein such as humanized E2 derived from the hepatitis C virus, (iii) a modified amino acid such as phosphatidylserine, (iv) an antibody configured against one or more extracellular domains of SCARB1 or CD36, or (v) a small interfering RNA configured against SCARB1 or CD36.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 depicts an immunohistochemical staining of advanced oxidation protein products (AOPP) and Ang II in sequential sections of renal biopsies taken from patients with kidney disease (IgA-nephropathy).

FIG. 2 is a graph showing that elevated AGE pentosidine levels correlate with decreased renal function.

FIG. 3 shows a Western blot and graph of renal proximal tubule cells (PTCs) treated with an AOPP for finite time periods, compared to a control and cells exposed to rat serum albumin (RSA). The y-axis on the graph is labeled “phospho-PKC (ratio to total)”.

FIG. 4 shows a graph of PTCs incubated with either a pan-PKC inhibitor (Gö6983) or PKCα inhibitor (Gö6976) before AOPP exposure. The y-axis on the graph is labeled “phospho-p47phox (ratio to total)”. The labels on the x-axis are, from left to right, “+Gö6983” and “+Gö6976”. The left white column and the leftmost black column are not labeled.

FIG. 5 shows a graph of intracellular reactive oxygen species (ROS) production related fluorescence intensity in renal PTCs after pre-incubation with various reagents and addition of AOPPs. The y-axis on the graph is labeled “Intracellular ROS production relative fluorescence intensity”. The labels on the x-axis are, from left to right, (1) “a-RAGE”; (2) “+NC siRNA”; (3) “+IgG”; (4) “+a-RAGE”; (5) “+si-CD36”; (6) “+si-CD36+a-RAGE”; (7) “+Gö6983”; and (8) “+Gö6976”. The left white column and the leftmost black column are not labeled.

FIG. 6 shows a graph of RAS protein level ratios relative to B-actin and a Western blot of oxidatively modified proteins after pre-incubation with various reagents and addition of AOPPs. The y-axis on the graph is labeled “protein levels of RAS (ratio to β-actin)”. The labels on the x-axis are, from left to right, (1) “+c-SOD”; (2) “+DPI”; (3) “+apocyanin”; (4) “+Gö6983”; and (5) “+Gö6976”. The two leftmost columns are not labeled.

FIG. 7 shows a graph of the level of phosphorylated PKCα (ratio to total), a regulator of p47phox phosphorylation, on the membrane of PTC cells treated with various reagents and AOPPs. The level of phosphorylated PKCα on the PTC cell membrane was determined by a Western blot. The y-axis on the graph is labeled “phospho-PKC (ratio to total)”. The labels on the x-axis are, from left to right, (1) “a-RAGE”; (2) “+NC siRNA”; (3) “+IgG”; (4) “a-RAGE”; (5) “+si-CD36”; and (6) “+si-CD36+a-RAGE”. The left white column and the leftmost black column are not labeled.

FIG. 8 shows a graph of the level of phosphorylated p47phox, a regulator of NADPH-oxidase activity, which was analyzed by co-immunoprecipitation. The y-axis on the graph is labeled “phospho-p47phox (ratio to total)”. The labels on the x-axis are, from left to right, (1) “a-RAGE”; (2) “+NC siRNA”; (3) “1-IgG”; (4) “a-RAGE”; (5) “+si-CD36”; and (6) “+si-CD36+a-RAGE”. The left white column and the leftmost black column are not labeled.

FIG. 9 shows a Western blot and graph of phosphorylated PKCα (ratio to total) in the renal cortex of rats subjected to either unilateral nephrectomy (UNX) or to sham operation (sham). Various reagents were injected into the rats a week after the operations. The y-axis on the graph is labeled “phospho-PKC (ratio to total)”.

FIG. 10 shows a Western blot and graph of the protein levels of membrane-associated p47phox (ratio to sham) in the renal cortex of rats subjected to either unilateral nephrectomy (UNX) or to sham operation (sham). Various reagents were injected into the rats a week after the operations. The y-axis on the graph is labeled “protein levels of p47phox (ratio to sham)”.

FIG. 11 shows a graph of superoxide generation in rat renal cortex homogenates as detected by lucigenin chemiluminescence method. The y-axis on the graph is labeled “superoxide generation (counts/min/100 μg protein)”.

FIG. 12 shows a graph of protein levels of AP-1 (ratio to β-actin), along with expression of its subunits c-jun and c-fos, and the phosphorylation of c-jun, in rat renal cortex, as evaluated by Western blot. The y-axis on the graph is labeled “protein levels of AP-1 (ratio to β-actin)”. AP-1 is a nuclear receptor stress-response protein and inducer of apoptosis.

FIG. 13 shows a graph of the NF-κβ subunit phospho-p65 (ratio to total) in rat renal cortex, as evaluated by Western blot. The y-axis on the graph is labeled “phospho-p65 (ratio to total)”.

FIG. 14 is a set of two pictures showing a cross-section of arteries from a healthy kidney donor (left-hand side) and a patient undergoing renal transplantation (right-hand side).

FIG. 15 is a set of stained sections from two patients undergoing renal transplantation (P1, P2) and one healthy kidney donor as control (C). Most notable is the lack of MDA expression in the control.

FIG. 16 is a graph showing levels of phosphorylated PKCα expression between patients and controls.

FIG. 17 is a graph showing levels of p47 expression between patients and controls.

FIG. 18 is a graph showing levels of SCARB1 expression between patients and controls.

FIG. 19 is a graph showing levels of reactive oxygen species (ROS) production in vascular endothelial cells with varying time exposures to an AOPP or AGE.

FIG. 20 is a graph showing levels of ROS production with pre-incubation to antibody or siRNA and then exposure to an AOPP or AGE.

FIG. 21 is a bar graph showing the expression levels of angiotensin (AGT), angiotensin-converting enzyme (ACE), and Angiotensin II receptor type 1 (AT1) after pre-incubation with antibody or siRNA and then exposure to an AOPP or AGE.

### DETAILED DESCRIPTION

A more complete understanding of the compositions and methods disclosed herein can be obtained by reference to the accompanying drawings. These figures are merely schematic representations based on convenience and the ease of demonstrating the present disclosure, and are, therefore, not intended to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

All publications, patents, and patent applications discussed herein are hereby incorporated by reference in their entirety.

Knowledge of the fate of circulating signalling peptides following receptor binding remains incomplete, even though endocrine signalling using peptide hormones—eg. insulin, interleukins and shed surface proteins such as sRAGE—takes place along some of the most studied pathways in humans. It is generally assumed that the peptides dissociate from their receptors and are subsequently broken down. However, recent evidence identifying a novel role for insulin degrading enzyme (IDE) in the proximal renal tubule, where endocytosis and lysomal degradation determine the clearance of many peptide hormones, suggests a more complex fate. Clinically, more than 30% of patients with insulin-dependent (IDDM) or non-insulin dependent (NIDDM) diabetes mellitus have chronically reduced renal function, and insulin resistance is reported to be exceedingly common in patients with chronic kidney disease (CKD), regardless of etiology.

Mortality from acute kidney injury (AKI) or chronic kidney disease (CKD) is linked to the degree of renal function loss, or lost ability to perform glomerular filtration. In this regard, the kidney is responsible for clearing circulating proteins, especially low molecular-weight proteins that pass over the glomerular filter, such as insulin and glucagon. Recent studies have demonstrated that following depletion of albumin (which is low in CKD), circulating levels of most blood proteins are elevated in kidney disease and inversely correlate with renal function, likely due to the impact of reduced renal clearance. Highly elevated circulating levels of most cytokines, as well as of partly metabolized fragments of several hormones, including parathyroid hormone (PTH), prolactin, and glucagon-like peptides 1 and 2, amongst others, have been found in individuals with reduced glomerular filtration. Data indicate that these increased concentrations are proportional to a prolonged half-life of plasma proteins in AKI and CKD. It appears that following receptor binding and activation, most peptide hormones disengage from their specific receptors. Many are then cleaved by peptidases into inactive polypeptides, while the vast majority of both uncleaved hormones and polypeptides soon pass over the renal glomerular filter (being smaller than the filter cut-off of approximately 7 nanometers to 8 nanometers) and are metabolized in the renal tubules, mainly but not exclusively by LRP2/CUB-receptor endocytosis.

Multiple reports have identified biological activity in circulating, post-secretory modified peptide hormones. Molecules reported to possess such activity include peptidase-cleaved insulin, IGF-I, and IGF-II, along with phosphorylated incretins, whose affinity for their receptors was actually found to be greater than that of non-phosphorylated, newly synthesized incretins. It is now clear that oxidative modification of peptide hormones leads to changed affinity for both their respective receptors as well as for circulating peptidases.

The demonstration that oxidatively modified forms of proteins accumulate during aging, oxidative stress, and in some diseases has focused attention on the modification of biological molecules by various kinds of free radical species (ROS), defined as clusters of atoms one of which contains an unpaired electron in its outermost electron orbit. This is an extremely unstable configuration, and such radicals quickly react with other molecules or radicals to achieve a more stable configuration. Examples of ROS include hydrogen peroxide, hypochlorite ion, hydroxyl radical or superoxide anion. AKI and CKD are characterized by very high levels of ROS. These ROS oxidatively modify the proteins which have accumulated after failure of the kidney to perform sufficient filtration. Diabetes mellitus is another such condition characterized by very high levels of ROS.

There are three predominant groups of ROS-modified proteins. The first group is advanced oxidation protein products (AOPPs), which are formed by the reaction of oxidative radicals with protein amino acids. The second group is advanced glycation end-products (AGEs), which are formed by the reaction of proteins with carbonyl groups generated by earlier oxidation of lysine, arginine, proline, and threonine resides, or during the reduction of sugars. The third group is phosphorylated protein residues (PPRs), which result from native kinase activity that may be amplified by previous oxidative protein modification.

AOPPs have been reported to bind to the transmembrane Receptor for Advanced Glycation Endproducts (RAGE), cluster of differentiation (CD)36, as well as the scavenger receptor class B type I (SCARB1, also known as CD36L1, CLA1, SRB1, or SR-BI). AGEs have been reported to bind to RAGE and EN-RAGE (a co-receptor implicated in mediating inflammatory signaling), and to bind to CD36 and AGE-R 1-3. PPRs may bind their specific receptor with altered affinity, along with one or more scavenging receptors recognizing PPR motifs, such as SCARB or LRP2.

Pathways downstream of these receptors are believed to cause damage to the body. In mice, reducing oxidative protein damage without energy or nutrient change improves host defenses, alleviates inflammation, prevents diabetes mellitus, vascular and renal complications, resulting in an extended lifespan. Studies in healthy humans and patients with diabetes mellitus have also linked consumption of AGEs to insulin resistance and inflammation, while restricting AGE improves both. Some of the post-translational modifications described above also engender proteins with a greater affinity for formation of amyloid (protein aggregates). In patients with atherosclerosis but normal kidney function, a pro-oxidative environment has been reported to induce apolipoprotein amyloidosis.

Accumulating evidence suggests that reducing oxidative protein damage can improve AKI and CKD complications, as well as result in an extended lifespan. Animal models of high oxidative stress and the human phenotype of CKD are highly similar, providing indications of the negative downstream signaling effects which occur when AOPPs or AGEs bind RAGE, SCARB1, and/or CD36 receptors. Both animal models and human CKD patients suffer from RAS-activated hypertension, dyslipidemia, endothelial dysfunction, insulin resistance, and a pro-inflammatory activation. Both groups also suffer from endovascular remodeling with the accumulation of atherosclerotic plaques and vascular calcification. Mice with CKD overexpressing EN-RAGE develop vascular lesions similar to those seen in CKD patients. Such changes can be blocked by inhibiting NADPH-oxidase or RAGE. Also, RAGE −/− mice are immune to the nephrotoxic effects of a high-AGE diet, while rats with CKD exhibit a 3-fold increase in superoxide anion production from NADPH oxidase in the left ventricle, associated with enhanced expression of osteopontin and accumulation of pro-collagen type I.

Both AOPP-induced and AGE-induced RAGE signaling have been implicated in the pathophysiology of multiple renal disorders including obesity-related glomerulopathy, doxorubicin-induced nephropathy, hypertensive nephropathy, lupus nephritis, renal amyloidosis, and ischemic renal injuries. RAGE-mediated effects through downstream NADPH-oxidase, NF-Kβ, and RAS pathways have also been implicated in arterial calcification, inflammation, and endothelial dysfunction in CKD patients. Endogeneous ROS also upregulates Nrf2 activity to activate antioxidative defenses, while the Nrf2-inducer bardoxolone was recently shown to be nephroprotective in patients with type-2 diabetes. As a result, it is believed that an effective treatment of renal failure is to reduce the downstream effects of AOPP-induced and/or AGE-induced RAGE, SCARB1, or CD36 signaling.

In addition, it is believed that decreased renal clearance of plasma proteins increases the time of exposure of those plasma proteins to reactive oxygen species (ROS) and phosphorylating enzymes. This results in an increase in the formation of advanced oxidation protein products (AOPPs), advanced glycation end-products (AGEs), and phosphorylated protein residues (PPRs). The decreased renal clearance also reduces the rate at which AOPPs and AGEs are cleared from the body.

In one investigation of patients with reduced renal function using size-exclusion chromatography, greatly increased oxidation of proteins with a size of <80 kDa, i.e. the presence of AOPPs, was found. However, no AOPPs were found in the control group. In the same study, plasma levels of AOPP were highest in patients on dialysis, followed by those with advanced CKD. Later studies have also reported increased plasma concentration of AOPPs in renal patients with diabetic nephropathy. Recent studies that looked at PPRs reported that it is serine-specific phosphorylation that is increased in patients with reduced renal filtration. This is of special interest as phosphorylation of serines often inhibits cleavage by peptidases.

Oxidized proteins may bind to semi-specific scavenging receptors by sharing common structures with oxidized cell surface proteins from apoptotic cells. Oxidized LDL (OxLDL) is known to be cleared through scavenger receptors, including CD36, LOX-1 and SCARB1. In studies, it partly competes with oxidatively damaged and apoptotic cells, implying the presence of one or more common domains. Evidence suggests that oxidized phospholipids, present in OxLDL and also in the membrane of apoptotic cells, represent one such common domain. These oxidized phospholipids, either in the lipid phase of OxLDL or becoming attached covalently to apoprotein B during LDL oxidation, have been shown to play a major role in the binding of OxLDL to CD36 and to SCARB1 expressed in transfected cells. The lipid and protein moieties compete with each other to some extent, indicating that they are binding to at least one common site. A monoclonal antibody against oxidized phospholipids (but not native phospholipids) was able to block the uptake of OxLDL by CD36 in transfected cells by as much as 80%; it also inhibited macrophage phagocytosis of apoptotic cells by about 40%. Thus, the persistence of receptors for OxLDL during evolution is probably accounted for by their role in recognition of ligands on the surfaces of oxidatively damaged or apoptotic cells.

The present disclosure shows that the cell-surface receptor proteins SCARB1 and CD36 are critical for binding and internalizing oxidatively modified proteins, such proteins being present in increased quantities in patients with reduced glomerular filtration where they are associated with tissue amyloid, RAS activation, and enhanced ROS production. Treatment of renal failure and its vascular complications can therefore utilize the specific ability of SCARB1 and/or CD36 to bind oxidatively modified proteins to remove those proteins with an extracorporeal device. Treatment may alternatively proceed by inhibiting SCARB1 and/or CD36 function through the use of an antagonist drug against one or both receptors. Such drugs may inhibit the normal SCARB1 and/or CD36 binding function or signaling to reduce the subsequent downstream effects that exacerbate kidney disease and lead to some of its' complications.

It has also been discovered that patients with reduced renal function (especially including diabetic nephropathy) may accumulate amyloid (insoluble fibrous protein aggregates or crystals, not necessarily formed by the protein SAA) outside of the central nervous system, especially in the vascular endothelium. Indeed, it is believed that uptake of modified proteins into endocytosis-competent or phagocytosis-competent cells, especially in the perivascular space, facilitates amyloid fibril growth. These cells subsequently undergo cell death and intracellular amyloid structures become released into the extracellular space. It has also been discovered that the scavenger receptor class B, subtype 1 (SCARB1 or SR-B1) may play multiple roles in reference to this. SCARB1 mediates the clearance of oxidatively modified proteins, especially proteins alkylated by malondialdehyde (MDA). SCARB1 also mediates the clearance of proteins where one or more amino acids have reacted with the Maillard reaction products of ribose to form the advanced glycation endproduct (AGE) pentosidine. This can be important when the main clearance pathway through the kidney is impaired. Finally, it appears that oxidatively modified proteins accumulate in cells that express SCARB1. This accumulation can facilitate amyloid formation, which is known to cause cell damage and apoptosis. This information can be used for multiple applications, e.g. diagnosis, risk stratification, therapy monitoring, and drug therapy.

In some embodiments disclosed herein, a treatment for complications of renal failure is a process comprising contacting a patient's blood with a filter body. The filter body includes an immobilized CD36 receptor, a CD36 receptor fragment, an SCARB1 receptor, and/or an SCARB1 receptor fragment. Alternatively, the filter body may include an antibody against an amino-acid sequence (motif) found in all CD36 or SCARB1 ligands, and whose recognition by the receptor is necessary for normal binding thereto. Due to the binding of CD36/SCARB1 to oxidatively modified proteins, e.g. AOPPs and AGEs, such proteins bind to the filter body, and the patient's blood will have a reduced quantity of oxidatively modified proteins after contacting the filter body.

The terms “modified proteins” or “oxidatively modified proteins” as used herein denotes soluble polypeptides that are derived from an originally secreted protein present also in healthy individuals. As disclosed herein, in patients with kidney dysfunction these original proteins undergo modifications in the circulation leading to the formation of advanced oxidation protein products (AOPPs), advanced glycation end products (AGEs) and/or phosphorylated protein remnants (PPRs). A “modified protein” is here defined as one that belongs to one or more of AOPPs, AGEs and PPRs.

The term “CD36 receptor” is used herein to refer to the integral membrane protein which is a member of the Class B scavenger receptor family (SCARB3). In humans and in mice, CD36 consists of 472 amino acids. The full sequence for human CD36 is provided as SEQ ID NO: 1. The term “CD36 receptor fragment” refers to a fragment of the CD36 protein that retains at least one function of the protein. For example, residues 30-439 form an extracellular topological domain. Residues 93-120 are required for binding to thrombospondins. Residues 87-99 are a PKC phosphorylation site. Residues 28-93 and 155-183 are involved in the binding of oxLDL. The domain between amino acids 139-184, amino acids 146-164, and amino acids 145-171 have also been shown to mediate binding with PfEMP-1.

The term “SCARB1 receptor” is used herein to refer to the integral membrane protein which is a member of the Class B scavenger receptor family (SCARB1). The full sequence for human SCARB1 has 552 amino acids, and is provided as SEQ ID NO: 2. The term “SCARB1 receptor fragment” refers to a fragment of the SCARB1 protein that retains at least one function of the protein. For example, residues 33-443 form an extracellular topological domain.

The term “amyloid precursor” is used to refer to ROS-modified proteins or polypeptides that accumulate in renal failure and are able to form aggregates but have not yet done so. This differentiates them from amyloid, which are insoluble fibrous protein aggregates or crystals, not necessarily formed by the protein SAA or its' cleavage products. Modified proteins are examples of amyloid precursors.

The term “filter body” refers to a surface on which the receptor or receptor fragment can be immobilized. The filter body may be of any suitable shape. For example, the filter body may comprise beads or particles, such as microparticles or nanoparticles; hollow fibers; or a membrane. Other forms of filter bodies include a porous bead, a mesh, or a bag (similar to a “tea bag”) where the receptor or receptor fragment is immobilized on an inner surface thereof. These may be incorporated into a larger housing. For example, beads may be used in a column.

The term “immobilized” means that the receptor or receptor fragment has been purposefully attached to the surface of the filter body. The immobilization may be indirect, such as using well-known affinity systems. Examples include the interaction between a His-tag in the protein molecule and the filter body provided with a chelating moiety such as Ni-NTA groups (or vice versa), or the interaction between biotin and streptavidin. The immobilization may also be direct, i.e. the protein molecule being covalently attached to the surface of the filter body. Exemplary covalent attachments include covalent polymer grafting, plasma treatment, physisorption, chemisorption and chemical derivatization. These and other methods and means for immobilization are contemplated.

In some embodiments, the material of the filter body may be selected from the group consisting of glass, cellulose, cellulose acetate, chitin, chitosan, cross-linked dextran, cross-linked agarose, agar gel support, polypropylene, polyethylene, polysulfone, polyacrylonitrile, polytetrafluoroethylene, polystyrene, polyurethane, silicone and amylase coated particles. A filter body which is cheap and easy to manufacture and handle but keeps leakage to a minimum is advantageous.

The filter body can be placed in a housing. In some embodiments, the housing is in the form of a shell which comprises a fluid inlet, a fluid outlet and a chamber in which the filter body is placed.

If desired, the housing can include a size exclusion filter. Filtration is a mechanical or physical operation which is used for the separation of solids by interposing a medium through which only selected solids can pass. A size exclusion filter allows only molecules of a given size to pass through the filter, and retains molecules above the given size. Mesh, bag and paper filters may be used to remove large particulates suspended in fluids while membrane processes, including microfiltration, ultrafiltration, nanofiltration, reverse osmosis and dialysis, employ synthetic membranes and may be used to separate micrometer-sized or smaller species. A size exclusion filter may have various forms, such as a fiber, a perforated sheet or a mesh type filter. The size exclusion filter can be made of a natural material, such as cellulose or a derivative thereof, chitosan, carbon or aluminium oxide. The size exclusion filter can alternatively be made from a man-made material such as nylon 6-6, polyvinylidene fluoride, polypropylene, polytetrafluoroethylene, polyethersulfone, glass and metal. Ideally, the filter is dimensioned so as to prevent the passage of blood proteins that in their unmodified state normally do not pass the glomerular filtration barrier.

If desired, the housing can include a charge filter. A charge filter is characterized by having a stable electric charge, such as between −0.005 C/m² and −0.019 C/m². Again, a charge filter may have various forms such as a fiber, a perforated sheet or a mesh type filter. The charge filter can be made of a natural material, such as cellulose or a derivative thereof, chitosan, carbon or aluminium oxide. In other examples, the charge filter can be made from a man-made material such as nylon 6-6, polyvinylidene fluoride, polypropylene, polytetrafluoroethylene, polyethersulfone, glass and metal. Ideally, the filter is charged so as to prevent the passage of blood proteins that in their unmodified state normally do not pass the glomerular filtration barrier.

In some embodiments, the size exclusion filter and the charge filter is the same filter. In some examples, the size exclusion filter and the charge filter are two different filters. For example, the size exclusion filter may be placed before the charge filter in the housing. In this example, the biological fluid added to the housing reaches the size exclusion filter before the charge filter. In other examples, the charge filter is placed before the size exclusion filter. In yet other examples, the size exclusion filter is located upstream of the filter body.

The filter body is then contacted with a biological fluid. It is contemplated that the CD36/SCARB1 receptor(s), CD36/SCARB1 receptor fragment(s), or antibody(s) against CD36/SCARB1-binding motifs on the filter body will bind to modified proteins present in the biological fluid. These modified proteins are retained by the filter body and separated from the biological fluid, reducing their concentration in the filtered fluid. The filtered fluid (e.g. blood) can then be returned to the patient in a cleaner state.

The term “biological fluid” refers to any fluid which can be obtained from an animal (e.g. mouse, human) or contains a biological molecule. For example, a biological fluid can be any water-based fluid comprising for example diverse solutes, suspended naturally occurring or manufactured polypeptides and cells. For example, the biological fluid may be blood, which contains proteins, salts and other molecules. In other examples, the biological fluid is plasma, serum or urine.

The term “plasma” refers to the yellow liquid component of whole blood, in which the blood cells in whole blood would normally be suspended. Put another way, plasma is whole blood minus the blood cells. Plasma is mostly water and comprises dissolved proteins, glucose, clotting factors, mineral ions, hormones and carbon dioxide. Plasma may be prepared by spinning a tube of fresh blood containing an anti-coagulant in a centrifuge until the blood cells fall to the bottom of the tube. The plasma is then poured or drawn off from the blood cells.

The term “serum” refers to plasma without clotting factors (i.e. whole blood minus both the blood cells and the clotting factors). Serum includes all proteins not used in blood clotting and all the electrolytes, antibodies, antigens, hormones, and any exogenous substances (e.g., drugs and microorganisms).

As a result of removing the modified proteins from the biological fluid, it is believed that complications of renal failure may be reduced. With lower levels of AOPPs and AGEs, the RAGE, CD36 and SCARB1 receptors in the body are less likely to be activated, and thus the detrimental downstream effects that cause cellular stress should be reduced. Also, the decreased number of modified proteins means that amyloid formation is retarded, and may be reversed.

In other different processes contemplated by the present disclosure, complications of renal failure can be treated by administering an antagonist drug to the patient. The antagonist drug blocks the natural RAGE receptors, the natural CD36 receptors or the natural SCARB1 receptors in the body. This prevents the AOPPs and AGEs from activating the downstream pathways that result in increased cellular stress.

An “antagonist drug” is a drug that does not provoke a biological response itself upon binding to a receptor, but blocks or dampens agonist-mediated responses. Antagonists have affinity but no efficacy for their cognate receptors, and their binding will disrupt the interaction and inhibit the function of an agonist or inverse agonist at the receptor. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding. Put another way, the antagonist drug competitively binds to such receptors to prevent their activation.

Exemplary antagonist drugs that are suitable for blocking the RAGE receptor include glitazones, FPS-ZM1, and low anti-coagulant 2-O,3-O-desulfated heparin (ODSH). ODSH is commercially available from Scientific Protein Laboratories (Wanaukee, Wis.). FPS-ZM1 has the structure shown below:

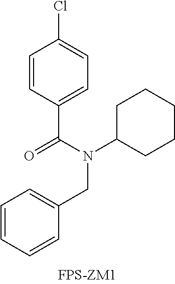 [SMILES: O=C(c1ccc(Cl)cc1)N(Cc1ccccc1)C1CCCCC1]

The glitazones are a class of medicines that contain a thiazolidinedione moiety. Exemplary members of this class include rosiglitazone, pioglitazone, troglitazone, netoglitazone, rivoglitazone, and ciglitazone, which are depicted below:

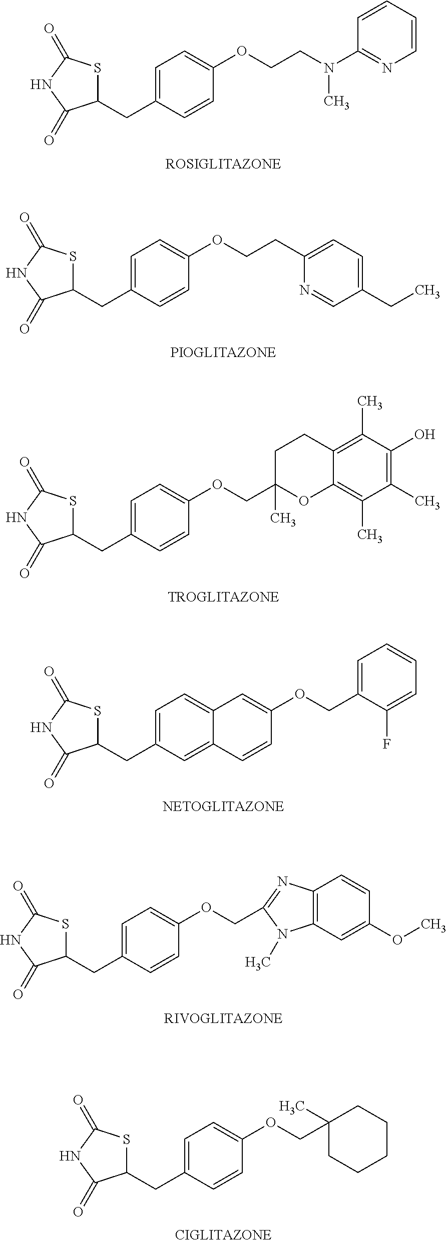 [SMILES: CN(CCOc1ccc(CC2SC(=O)NC2=O)cc1)c1ccccn1 ; CCc1ccc(CCOc2ccc(CC3SC(=O)NC3=O)cc2)nc1 ; Cc1c(C)c2c(c(C)c1O)CCC(C)(COc1ccc(CC3SC(=O)NC3=O)cc1)O2 ; O=C1NC(=O)C(Cc2ccc3cc(OCc4ccccc4F)ccc3c2)S1 ; COc1ccc2nc(COc3ccc(CC4SC(=O)NC4=O)cc3)n(C)c2c1 ; CC1(COc2ccc(CC3SC(=O)NC3=O)cc2)CCCCC1]

Exemplary antagonist drugs that are suitable for blocking the CD36 receptor include AP5055, AP5258, ursolic acid and derivatives thereof, and hexarelin.

Hexarelin is a peptide containing six amino acids. In particular, a methyl group is added to the second tryptophan residue. Hexarelin is CAS#140703-51-1 and is also known as L-Histidyl-2-methyl-D-tryptophyl-L-alanyl-L-tryptophyl-D-phenylalanyl-L-lysinamide. Hexarelin is illustrated below:

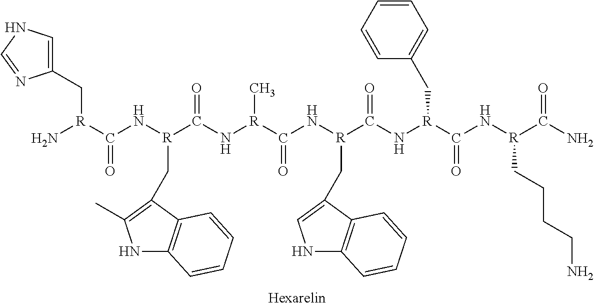

AP5055 and AP5258 are illustrated below:

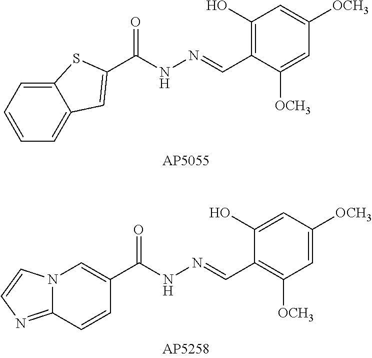 [SMILES: COc1cc(O)c(/C=N/NC(=O)c2cc3ccccc3s2)c(OC)c1 ; COc1cc(O)c(/C=N/NC(=O)c2ccc3nccn3c2)c(OC)c1]

Ursolic acid and its derivatives as contemplated herein may be illustrated below as Formula (A):

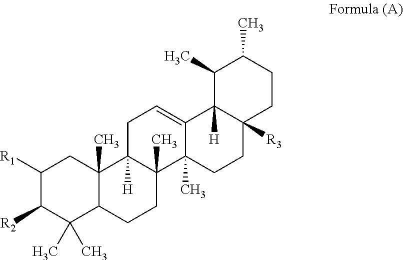

wherein R₁ is —H or —OH; R₂ is selected from —OH, —NH₂, and —O(CO)CH₃; and R₃ is selected from —COOH, —COOR, and —CONR^{a}R^{b}, wherein R is alkyl containing from 2 to 65 carbon atoms, wherein R^{a} and R^{b} are independently —H, —(CH₂)ₘNH₂ wherein m is 1 to 10, or —(CH₂)_{q}OH, wherein q is 1 to 3. Ursolic acid is the base compound, where R₁ is H, R₂ is —OH, and R₃ is —COOH.

Other suitable drugs are an anti-RAGE antibody or an anti-CD36 antibody, a small interfering RNA configured for silencing RAGE or CD36, a synthetic peptide or fusion protein, or a modified amino acid such as phosphatidylserine.

In some embodiments, the drug includes both (1) a small interfering RNA configured for silencing RAGE or CD36; and (2) either an anti-RAGE antibody or an anti-CD36 antibody.

In additional processes of the present disclosure, the binding of CD36 or SCARB1 to oxidative proteins can be targeted, modulated, or blocked. This can be a useful therapy for patients with reduced renal function. Controlling the binding of CD36 and/or SCARB1 can reduce oxidative damage to, and reduce pro-apoptotic signaling in, cells that express CD36 and/or SCARB1 (e.g. in the endothelium, blood, kidney, and eye) which otherwise accumulate oxidative proteins that damage these cells. This in turn reduces the renin-angiotensin-aldosterone system signaling (RAS) and oxidative stress that would otherwise occur in these and other cells due to said damage, thus slowing the progression of kidney disease and/or vascular damage associated with increased circulating levels of modified proteins. Reducing RAS activation also reduces the incidence of high blood pressure.

This can be achieved by administering an antagonist drug to the patient that blocks the natural CD36 and/or SCARB1 receptors, thus preventing modified proteins from binding to CD36 and/or SCARB1. Examples of such antagonist drugs suitable for blocking the CD36 receptor are listed above. Compounds suitable for blocking the SCARB1 receptor include acetyl-salicylate, sodium-salicylate, ezetimibe and similar cholesterol lowering agents, ITX-5061, ITX-7650, enfuvirtide, and peptides containing SCARB1 binding motifs but designed to resist internalization or receptor recycling. ITX-5061 is depicted below:

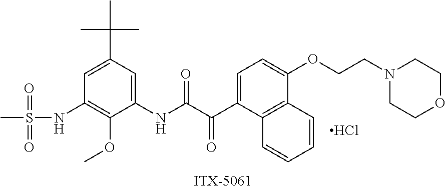 [SMILES: COc1c(NC(=O)C(=O)c2ccc(OCCN3CCOCC3)c3ccccc23)cc(C(C)(C)C)cc1NS(C)(=O)=O ; Cl]

Other suitable compounds for blocking the SCARB1 receptor are disclosed in EP 1991215, the entirety of which is hereby incorporated by reference herein. Compounds of Formulas (I)-(V) are disclosed therein, as listed below:

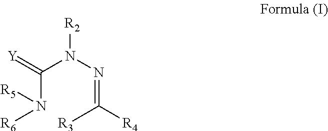

wherein R₂ is hydrogen or alkyl, optionally substituted;
R₃ and R₄ together form a cycloalkyl, heterocycloalkyl, cycloalkenyl or heterocycloalkenyl;
R₅ is hydrogen or alkyl, optionally substituted;
R₆ is hydrogen, hydroxyl, halogen, alkyl, cycloalkyl, heteroalkyl, cycloheteroalkyl, aryl, aralkyl, heteroaryl, heteroaralkyl, alkenyl, cycloalkenyl, alkynyl, alkanoyl, alkoxyalkyl;
or —COR′, wherein R′ is hydrogen, alkyl, cycloalkyl, alkenyl, cycloalkenyl, aryl, aralkyl, heteroalkyl, cycloheteroalkyl, heteroaryl, heteroaralkyl, or alkynyl,

### and Y is S or NH;

or pharmaceutically acceptable salts thereof.

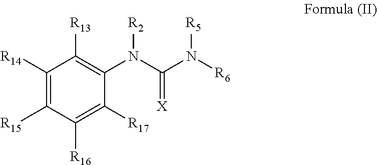

wherein R₂ is hydrogen or alkyl, optionally substituted;
R₅ is hydrogen, alkyl or alkenyl, optionally substituted;
R₆ is hydrogen, hydroxyl, halogen, alkyl, heteroalkyl, cycloalkyl, cycloheteroalkyl, aryl, aralkyl, heteroaryl, heteroaralkyl, alkenyl, cycloalkenyl, heteroalkenyl, cycloheteroalkenyl or alkynyl, optionally substituted; and
R₁₃, R₁₄, R₁₅, R₁₆, and R₁₇ are independently hydrogen, hydroxyl, halogen, SO₂, NO₂, CN, alkyl, heteroalkyl, cycloalkyl, heterocycloalkyl, alicyclic system, aryl, aralkyl, aralkenyl, aralkynyl, heteroaryl, heteroaralkyl, heteroaralkenyl, heteroaralkynyl, alkenyl, cycloalkenyl, alkynyl, or NR₁₁R₁₂, optionally substituted;
R₁₁ is hydrogen or alkyl, optionally substituted;
R₁₂ is hydrogen, hydroxyl, halogen, alkyl, heteroalkyl, aryl, aralkyl, heteroaryl, heteroaralkyl, alkenyl, cycloalkenyl, or alkynyl, optionally substituted;

### and X is S or O;

or pharmaceutically acceptable salts thereof;

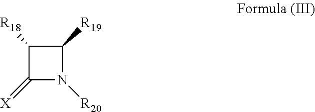

wherein R₁₈ is alkyl, alkenyl, aryl, aralkyl, heteroaryl, or heteroaralkyl, optionally substituted;
R₁₉ and R₂₀ are independently alkyl, alkenyl, aryl or heteroaryl, optionally substituted; and

### X is O or S;

or pharmaceutically acceptable salts thereof;

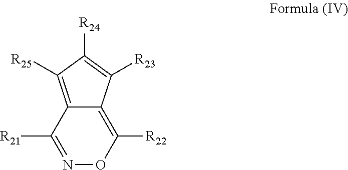

R₂₁ and R₂₂ are independently aryl, aralkyl, heteroaryl or heteroaralkyl, optionally substituted; and
R₂₃, R₂₄, and R₂₅ are independently hydrogen, hydroxyl, F, Cl, Br, I, CN, SO₂, NO₂, alkyl, heteroalkyl, aryl, aralkyl, heteroaryl, heteroaralkyl, alkenyl, cycloalkenyl, or alkynyl, optionally substituted;
or pharmaceutically acceptable salts thereof;

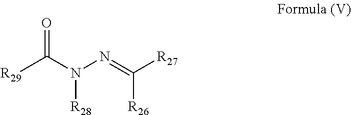

wherein R₂₆ is hydrogen, aryl, aralkyl, heteroaryl or heteroaralkyl, optionally substituted;
R₂₇ is aryl, aralkyl, heteroaryl or heteroaralkyl, optionally substituted;
R₂₈ is hydrogen or alkyl, optionally substituted; and
R₂₉ is aryl, aralkyl, heteroaryl or heteroaralkyl, optionally substituted;
or pharmaceutically acceptable salts thereof.

The term “alkyl” refers to a saturated straight or branched chain containing only carbon atoms and hydrogen atoms. Preferably, the chain comprises from 1 to 16 carbon atoms.

The term “heteroalkyl” refers to a saturated straight or branched chain containing carbon atoms and hydrogen atoms, wherein a carbon atom is replaced with a heteroatom. The heteroatoms are selected from O, S, and N. An exemplary heteroalkyl chain would be —CH₂—O—CH₃.

The terms “cycloalkyl” and “heterocycloalkyl” represent cyclic versions of “alkyl” and “heteroalkyl”, respectively, having a minimum of 3 carbon atoms. The terms “cycloalkyl” and “heterocycloalkyl” are also meant to include bicyclic, tricyclic and polycyclic versions thereof. If bicyclic, tricyclic or polycyclic rings are formed it is preferred that the respective rings are connected to each other at two adjacent carbon atoms, however, alternatively the two rings are connected via the same carbon atom, i.e. they form a Spiro ring system or they form “bridged” ring systems. Examples of cycloalkyl groups include cyclopropyl, cyclobutyl, cyclopentyl, cyclohexyl, cycloheptyl, spiro-[3,4]-octyl, bicyclo-[2.2.1]-heptyl, bicyclo-[2.2.2]-octyl, and adamantyl. Examples of heterocycloalkyl groups include piperidinyl, tetrahydrofuranyl, and pyrrolidinyl.

The term “aryl” refers to an aromatic monocyclic ring containing 6 carbon atoms, an aromatic bicyclic ring system containing 10 carbon atoms or an aromatic tricyclic ring system containing 14 carbon atoms. Examples are phenyl, naphthalenyl, and anthracenyl.

The term “aralkyl” refers to an alkyl moiety, which is substituted by aryl, wherein alkyl and aryl have the meaning as outlined above. An example is the benzyl radical.

The term “heteroaryl” refers to an aryl moiety wherein at least one of the carbon atoms is replaced by a heteroatom selected from 0, N and S. Examples are furanyl, thiophenyl, oxazolyl, isoxazolyl, pyrrolyl, imidazolyl, pyrazolyl, pyridinyl, pyrimidinyl, indolyl, isoindolyl, benzothiophenyl, benzimidazolyl, benzoxazolyl, indoxazinyl, quinolinyl, isoquinolinyl, quinoxalinyl, and quinazolinyl.

The term “heteroaralkyl” refers to an alkyl moiety, which is substituted with a heteroaryl substituent. An example is 2-alkylypyridinyl, 3-alkylpyridinyl, or 2-methylpyridinyl.

Similarly the terms “aralkenyl”, heteroaralkenyl”, “aralkynyl” and “heteroaralkynyl” refer to an alkenyl or alkynyl moiety as defined above, which is substituted by an aryl and heteroaryl moiety, respectively, as defined above.

The term “alkenyl” refers to an unsaturated chain of carbon atoms and hydrogen atoms that contains one or more double bonds. Preferably, the alkenyl chain comprises from 2 to 8 carbon atoms.

The term “heteroalkenyl” refers to an alkenyl chain wherein at least one of the carbon atoms is replaced by a heteroatom selected from 0, N and S.

The terms “cycloalkenyl” and “heterocycloalkenyl” refer to cyclic versions of “alkenyl” and “heteroalkenyl”. The terms “cycloalkenyl” and “heterocycloalkenyl” are also meant to include bicyclic, tricyclic and polycyclic versions thereof.

The term “alkynyl” refers to refers to an unsaturated chain of carbon atoms and hydrogen atoms that contains one or more triple bonds.

The various radicals may be substituted with one ore more halogen atoms, e.g. Cl, F, or Br. One preferred radical is the trifluoromethyl radical.

The term “pharmaceutically acceptable salt” refers to salts of the compounds described. Pharmaceutically acceptable salts include acid addition salts which may, for example, be formed by mixing a solution of choline or derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like.

Alternatively, drugs that ameliorate the effects of oxidatively modified proteins binding to one or both of CD36 and SCARB1 can be administered to the patient instead. These drugs may include RAS signaling pathway modulators such as enalapril, losartan or spironolactone, or be oxygen scavengers, NADPH oxidase inhibitors, pan-PKC inhibitors, or PKCα inhibitors.

Examples of oxygen scavengers are copper-zinc superoxide dismutase (CuZn-SOD), pterin derivatives such as biopterin and neopterin, flavonoids such as silibinin, 2,3-dimethyl-6(2-dimethylaminoethyl)-6H-indolo-(2,3-b)quinoxaline (also known as B220), N-acetylcysteine, and ascorbic acid.

Exemplary NADPH oxidase inhibitors include diphenyleneiodonium (DPI), apocynin, procyanidins such as delphinidin, Schisandrin B, and annexin peptide Ac2-26.

Examples of pan-PKC inhibitors are Gö6983 (CAS#133053-19-7 and having the chemical formula 2-[1-(3-Dimethylaminopropyl)-5-methoxyindol-3-yl]-3-(1H-indol-3-yl) maleimide), genistein, calphostin C, and GF109203X. Go6983 and GF109203X are depicted below:

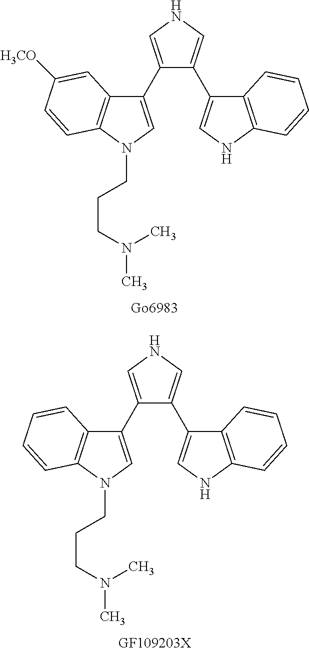 [SMILES: COc1ccc2c(c1)c(-c1c[nH]cc1-c1c[nH]c3ccccc13)cn2CCCN(C)C ; CN(C)CCCn1cc(-c2c[nH]cc2-c2c[nH]c3ccccc23)c2ccccc21]

Examples of PKCα inhibitors are Go6976 (CAS#136194-77-9 and having the chemical formula 12-(2-Cyanoethyl)-6,7,12,13-tetrahydro-13-methyl-5-oxo-5H-indolo(2,3-a)pyrrolo(3,4-c)-carbazole), PKCα (C2-4) inhibitor peptide, aprinocarsen, and MT477. Go6976 and MT477 are depicted below:

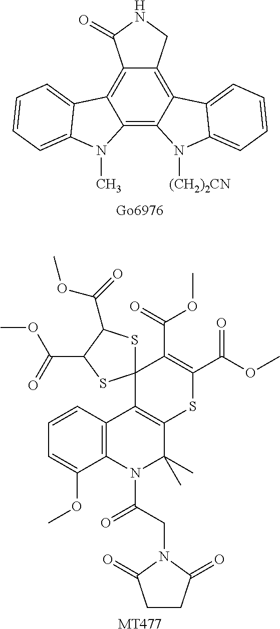

In other different processes contemplated by the present disclosure, complications of renal failure can be treated by administering a drug to the patient. The drug interferes with the formation of amyloid from amyloid precursors in the body, prevents cellular uptake of amyloid or its' precursors, and/or blocks one or more of the cytotoxic effects of amyloid.

Examples of drugs that interfere with the formation of amyloid are tramiposate, scyllo-inositol (AZD103), methylthioninium chloride (Rember™), metal ion modulators such as PBT2 (an 8-hydroxyquinoline copper/zinc ionophore), and GSK3β-inhibitors such as pyrazolopyrazines.

Examples of drugs that block the uptake of amyloid or its precursors into cells include the previously discussed antagonists of RAGE, CD36 and SCARB1; a synthetic peptide or fusion protein such as Na7 or humanized E2 derived from the hepatitis C virus; a modified amino acid such as phosphatidylserine; an antibody configured against one or more extracellular domains of RAGE or CD36 or SCARB1; and antibodies targeting RAGE/CD36/SCARB1 binding motifs or ROS-modifications such as AOPP on circulating proteins. Na7 is a peptide having the sequence EVHHQKL, and is SEQ ID NO: 3.

Exemplary drugs for ameliorating cytotoxic effects of amyloid/amyloid precursors include the previously enumerated oxygen scavengers, NADPH oxidase inhibitors, and PKC inhibitors, as well as ascorbic acid, amifostine and modulators of intracellular calcium concentration such as thapsigargin, icariin and CGP-37157. CGP-37157 is depicted below:

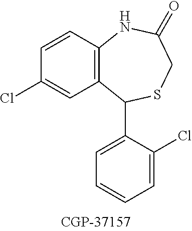 [SMILES: O=C1CSC(c2ccccc2Cl)c2cc(Cl)ccc2N1]

The drugs discussed herein are administered in an amount sufficient to obtain the desired effect.

It is also contemplated that patients at risk of vascular or other amyloid formation due to reduced kidney function can be identified or monitored by measuring the concentration of a specific molecule that binds to SCARB1, or the total concentration of all such molecules in a biological fluid. Such molecules may include malondialdehyde (MDA), AOPPs, or AGEs. This may be done for example by using a binding assay such as ELISA or immunofluorescence. Alternatively, the tissue amyloid content itself can be measured, or tissue RNA levels of CD36 or SCARB1 could be measured, or tissue protein levels of CD36 or SCARB1 could be measured. The measurement can be done by mixing a sample with a dye (e.g. Congo Red or pinacyanol), removing excess dye, and measuring the light absorbance spectrum or the reflectance spectrum of the tissue sample. The results can then be compared to a standard curve.

Another means of measurement would be to introduce a contrast agent to the sample, and measure the uptake of the contrast agent by the sample. Exemplary contrast agents include [¹⁸F]FDDNP, [¹¹C] Pittsburgh Compound B (PIB) as N-methyl [¹¹C] 2-(4′-methylaminophenyl)-6-hydroxy-benzothiasole, [¹¹C]SB-13 as 4-N-methylamino-4′-hydroxystilbene, [¹¹C]BF-227 as 2-(2-[-dimethylaminothizol-5-yl]ethenyl)-6-(2-[fluoro]benzoxazole), or [¹⁸F] BAY94-9172 as trans-4-(N-methylamino)-4A[spacing ring above]L-{2-[2-(2-[¹⁸F]fluoro-ethoxy)-ethoxy]-ethoxy}-stilbene.

### EXAMPLES

### Example 1

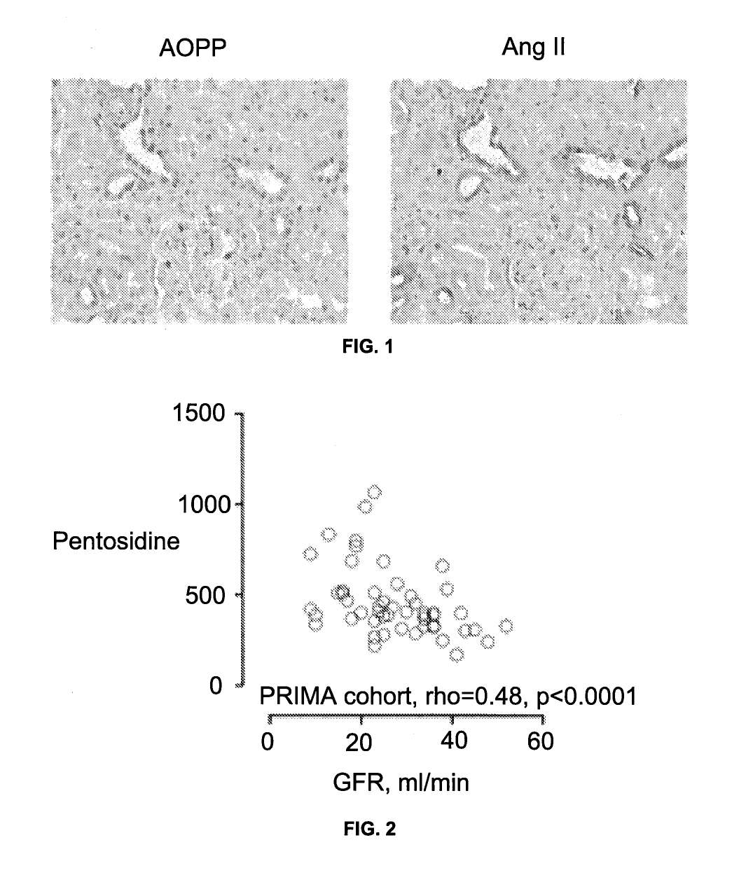

Renal biopsies from 19 human patients with IgA-nephropathy (aka Berger's disease) were examined using real-time PCR and Western blotting for the presence of AOPPs. It was found that AOPPs are mainly deposited in tubular epithelial cells also responsible for peptide endocytosis. Renal AOPP accumulation was co-localized with angiotension II (Ang II) and correlated with Ang II formation in renal tubules, suggesting that AOPPs may activate intrarenal RAS. FIG. 1 shows a representative image of an immunohistochemical stain for AOPPs (left) and a representative image of an immunohistochemical stain for Ang II (right). As can be seen here, they are generally found in the same locations. The correlation of renal AOPPs and Ang II staining in renal tubulointerstitium was (r=0.562, P<0.05).

### Example 2

FIG. 2 is a graph is shown indicating an inverse correlation between AGE accumulation levels and renal function. As glomerular filtration rate (GFR) increases, less pentosidine (an AGE) is present in the serum.

### Example 3

An experiment was performed that showed that advanced oxidation protein products (AOPPs) induce reactive oxygen species (ROS) production in cultured proximal tubule cells (PTCs) that is dependent on both PKCα and NADPH oxidase, mainly through CD36. Human Tubular Epithelial Cells (HUTEC) cells were cultured in DMEF/F-12 medium containing 10% fetal calf serum, penicillin (200 U/ml) and streptomycin (200 ug/ml) until confluence, and then left serum-free overnight.

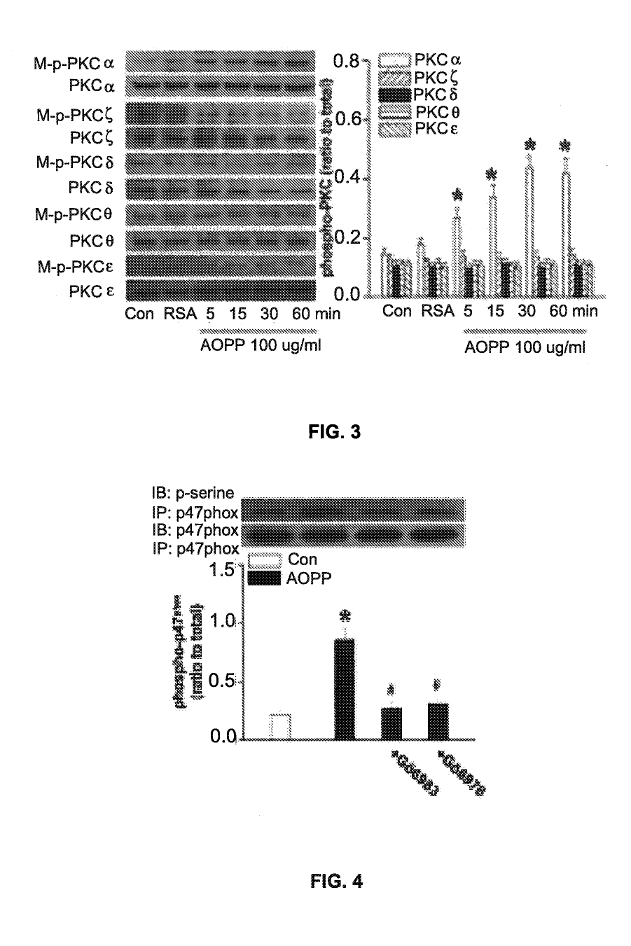

In sample set A, PTCs were treated with 100 micrograms/milliliter (μg/ml) of an AOPP (rat serum albumin treated with hydrochloric acid) or native rat serum albumin (RSA) for 5, 15, 30, or 60 minutes. Membrane protein was collected. The level of various phosphorylated protein kinase C isoforms (PKC) on the membrane was determined by Western blot. FIG. 3 shows the results. The “M-p-” designation indicates the amount of phosphorylated PKC, a regulator of NADPH-oxidase. The “Con” designation indicates the control cells, which were not treated at all.

In sample set B, PTCs were incubated with either a pan-PKC inhibitor (Gö6983) or PKCα inhibitor (Gö6976) before AOPP exposure. Phosphorylation of p47phox was assayed by immunoprecipitation. FIG. 4 shows the results. The results indicate that p47phox, a subunit of NAPDH oxidase which indicates the activation of this protein, is phosphorylated after AOPP addition, and that the p47phox is phosphorylated through pan-PKC or PKCα.

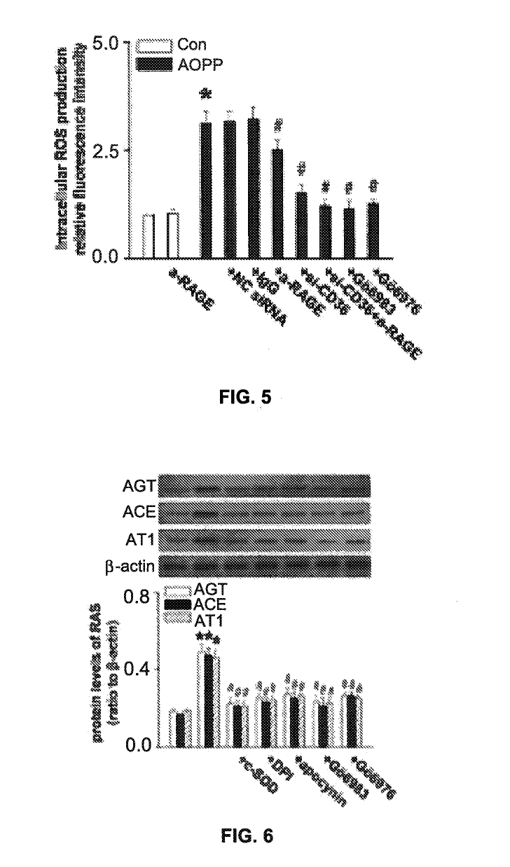

In sample set C, the PTC cells were pre-incubated with either a pan-PKC inhibitor (Gö6983), PKCα inhibitor (Gö6976), anti-RAGE antibody (a-RAGE), transfected with a CD36 siRNA (si-CD36), or both anti-RAGE antibody and transfected with CD36 siRNA (si-CD36+a-RAGE). The PTC cells were then exposed to AOPPs. Intracellular ROS production was determined by a fluorometric analysis. The results are shown in FIG. 5. This graph showed that lower levels of intracellular ROS production resulted in the PTC cells that were pre-incubated with CD36 siRNA alone. Further reduction was found in combination of the anti-RAGE antibody and CD36 siRNA, and in the PKC inhibitors.

In sample set D, the PTCs were treated with either copper-zinc superoxide dismutase (an O₂ scavenger, c-SOD), one of two inhibitors of NADPH oxidase (either diphenyleneiodonium (DPI) or apocynin), a pan-PKC inhibitor (Go6983), or PKCα inhibitor (Go6976). The PTCs were then exposed to 100 μg/ml of AOPPs. The protein levels of downstream renin-angiotensin-aldosterone (RAS) proteins were measured relative to β-actin. The expression of angioAGT, ACE, and AT1 was examined by Western blot. FIG. 6 shows the results. The lower levels of these proteins indicated that they were produced through an NADPH oxidase pathway, a pan-PKC pathway, and a PKCα pathway.

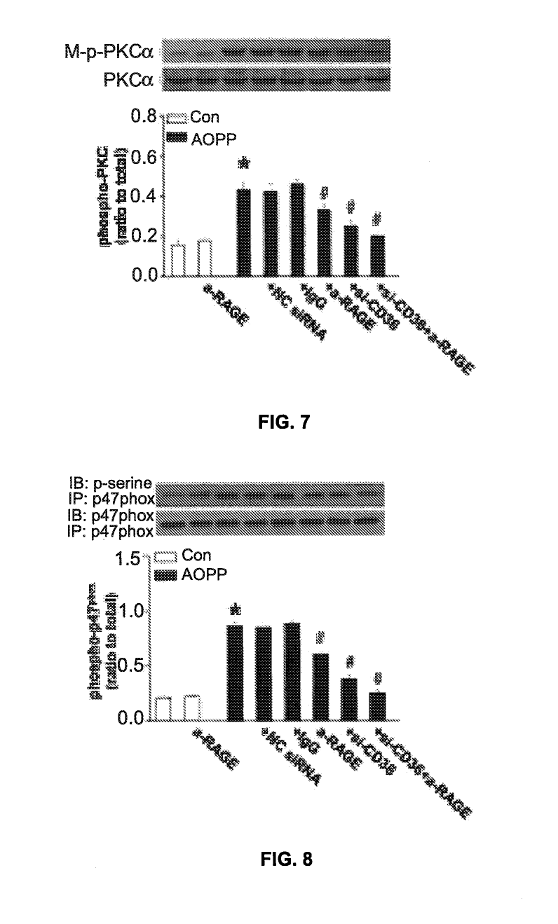

In sample set E, the PTCs were treated with either anti-RAGE antibody (a-RAGE), transfected with a CD36 siRNA (si-CD36), or both anti-RAGE antibody and transfected with CD36 siRNA (si-CD36+a-RAGE). The PTCs were then exposed to 100 μg/ml of AOPPs. The level of phosphorylated PKCα on the membrane was determined by Western blot. FIG. 7 shows the results. These indicate that blocking the RAGE or CD36 receptors can reduce phosphorylation of PKCα.

In sample set F, the level of phosphorylated p47phox in PTC cells was analyzed by co-immunoprecipitation. FIG. 8 shows the results. The same inhibitors that reduced the level of phosphorylated PKCα in FIG. 7 also reduced the phosphorylation of p47phox, implying a reduction in NADPH oxidase activity.

### Example 4

It has already been discussed that animal models of high oxidative stress and the human phenotype of CKD are highly similar. In addition to the PTC cell line experiments shown in FIGS. 3-9 and the human biopsy sample of FIG. 1, an experiment was performed in a rat renal cortex model to show that AOPP activates PKC, NADPH oxidase, AP-1 and NF-κB in rats with CKD. Rat renal cortex was subjected to either unilateral nephrectomy (UNX) or to sham operation (sham). One week after the operation, the UNX rats were randomized into 4 groups and received daily intravenous injection of endotoxin-free PBS, pH 7.4 (vehicle), native RSA (50 mg/kg per day) (Alb), AOPP-RSA (50 mg/kg per day) (AOPP), or a combination of AOPP-RSA (50 mg/kg per day) plus intragastric administration of apocynin (100 mg/kg per day, Sigma Chemical, St Louis, Mo., USA) (AOPP+apo).

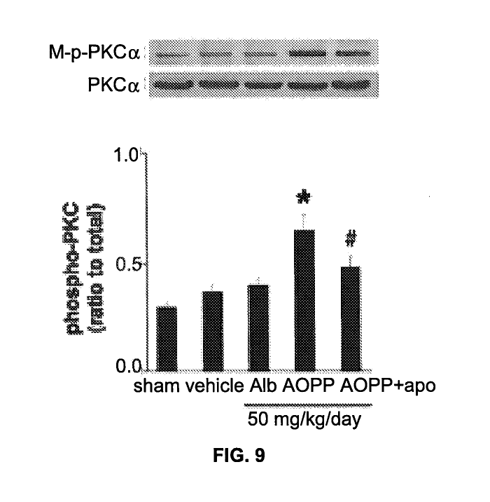

A Western blot was subsequently performed to determine the amount of membrane-associated phosphorylated PKCα after AOPP addition. The results are shown in FIG. 9. The rats exposed to AOPP had the highest levels of phosphorylated PKCα.

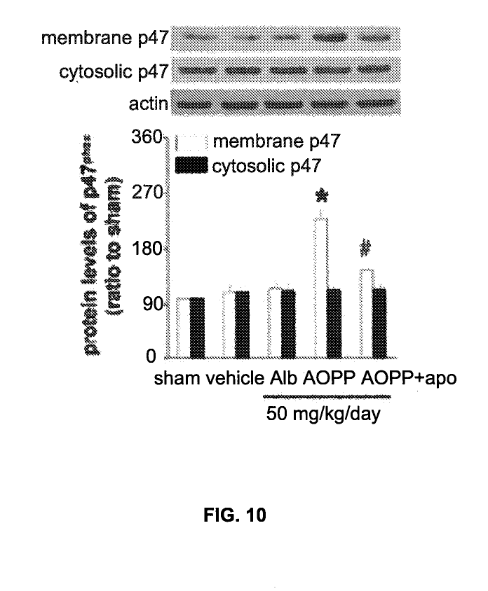

A Western blot was also subsequently performed to determine the protein levels of membrane-associated p47phox after AOPP addition. The results are shown in FIG. 10. Again, the rats exposed to AOPP had the highest levels, indicating that NADPH oxidase was activated.

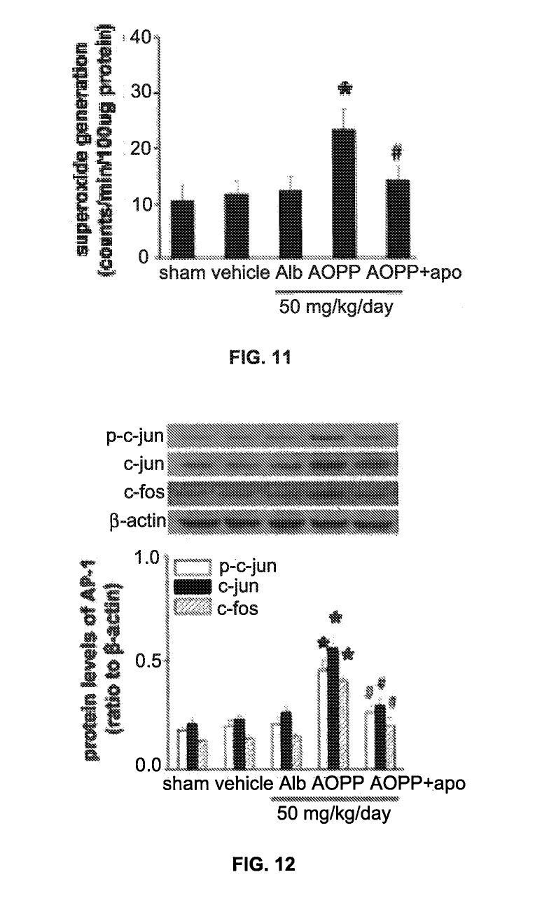

Next, superoxide (a reactive oxygen species) production in renal cortex homogenates was detected using the lucigenin chemiluminescence method. FIG. 11 shows the results. Again, the rats exposed to AOPP had the highest levels, indicating that AOPP exposure results in production of reactive oxygen species.

The effect of AOPP exposure on AP-1, a nuclear receptor stress-response protein and inducer of apoptosis, in the renal cortex was also measured. The proteins c-jun and c-fos (which combine to form AP-1) were separately measured. Phosphorylated c-jun was also measured as a sign of kinase activation. The results are shown in FIG. 12. Protein levels were highest in the rats exposed to AOPP.

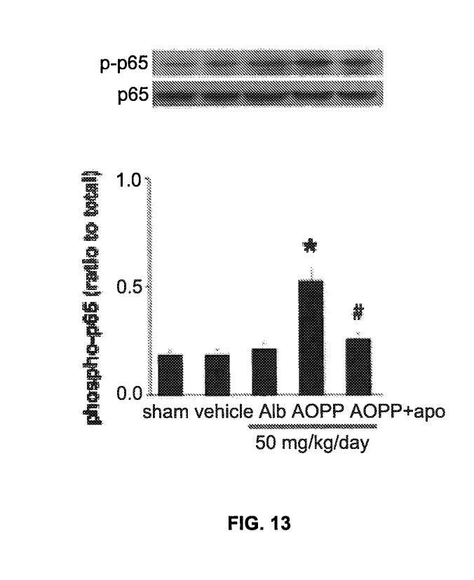

The phosphorylation of NF-KB p65 in renal cortex was also evaluated by Western blot and is shown in FIG. 13. Protein levels were highest in the rats exposed to AOPP.

In these experiments, β-actin was used to verify equivalent loading throughout. The ANOVA was P<0.05; *P<0.05 vs. vehicle, #P<0.05 vs. AOPPs-challenged group.

FIGS. 9-13 clearly indicate that AOPPs induce PKC, NADPH oxidase, AP-1, and NF-KB signaling pathway behavior in rats suffering from CKD. Given the similarity between animal oxidative stress models and the human CKD phenotype, similar results are expected to occur in humans as well. The processes of the present application are also expected to apply to both human and animals in the treatment of renal failure complications.

### Example 5

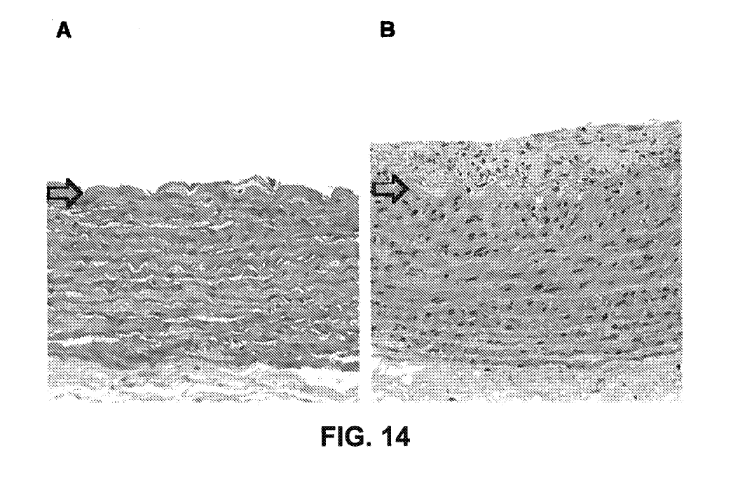

Biopsies were made of a. epigastrica from patients with end-stage renal disease undergoing renal transplantation. These were compared to biopsies taken from living kidney donors. Sections were mounted and stained with H&E (cells and calcium deposits) and Alcian blue (amyloid deposits). Two arterial cross-sections are shown in FIG. 14. The left-hand picture is from a healthy 38-year-old donor. The right-hand picture is from a 56-year-old patient on dialysis undergoing a renal transplantation. Amyloid deposits are in light green, with calcium deposits stained dark blue. Amyloid deposits are visible in the calcified intima in the right-hand picture.

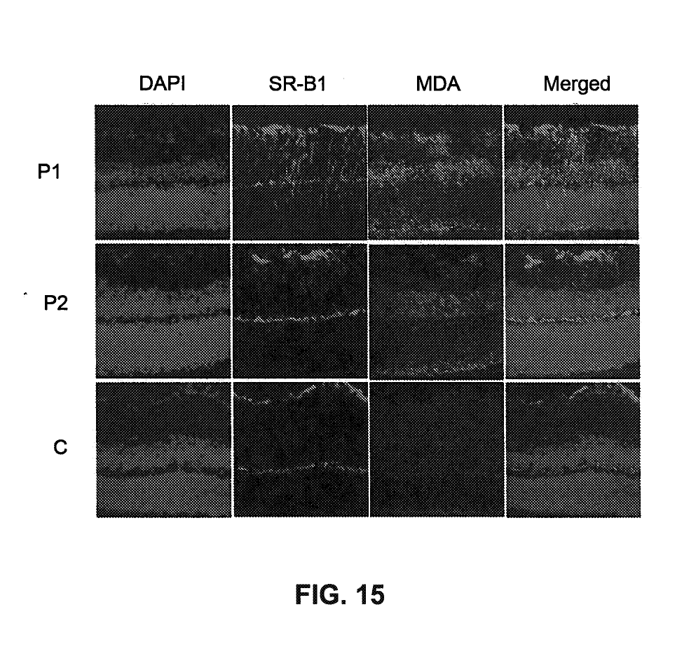

Next, immunofluorescent staining was performed on some sections. DAPI (blue) shows DNA. Antibodies to SCARB1 (red) were used, as were antibodies to protein-MDA (green). The sections for two patients with end-stage renal disease undergoing renal transplantation (P1, P2), and the sections for one control healthy live kidney donor (C) are shown in FIG. 15. These sections show that vascular amyloid is present in actual patients (but not in controls) and that it co-localizes with protein-MDA and SCARB1. There is increased SCARB1 staining in the two patients compared to the control, and the staining also goes deeper into the vessel wall, suggesting that the modified proteins have penetrated past the endothelial cell layer, and are present in the vascular media, where calcification is common in patients with CKD, and where one also finds a pathological amount of cells expressing SCARB1.

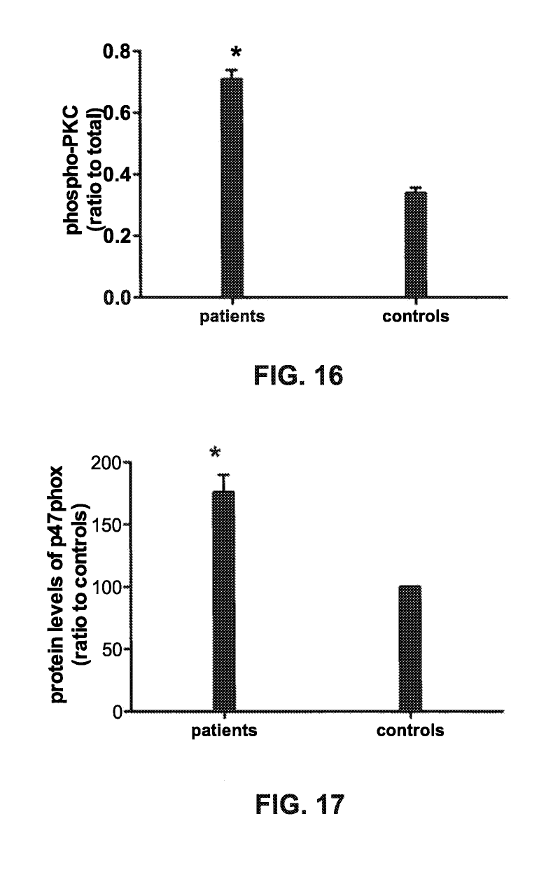

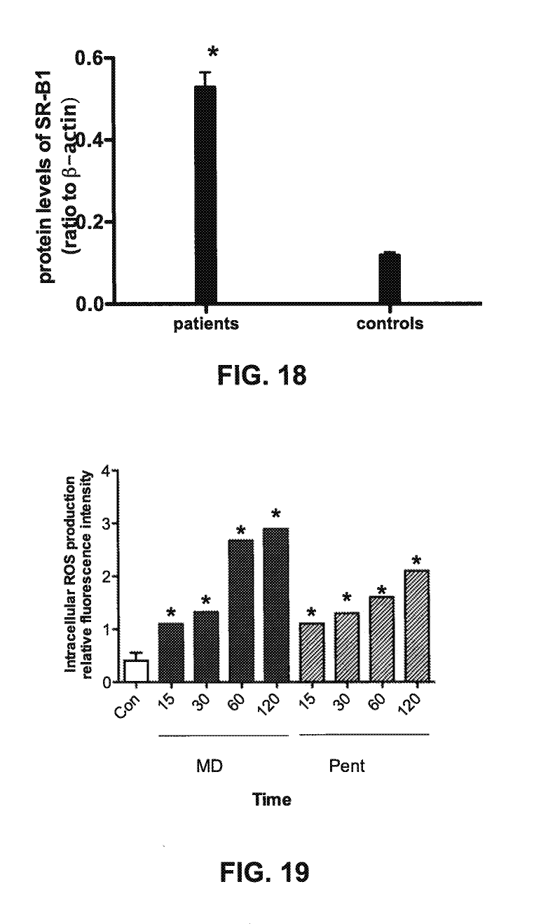

FIG. 16 is a graph showing the relative levels of phosphorylated PKCα between the patients and controls. FIG. 17 is a graph showing the levels of membrane and cytosolic p47 (a unit of NADPH oxidase) between the patients and controls. FIG. 18 is a graph showing the levels of SCARB1 between the patients and controls. N=3 in each group. The levels were higher for each compound in the patients compared to the controls.

### Example 6

Interfering with human microvascular endothelial cell (HMVECad) SCARB1 expression or blocking the receptor using an antibody ameliorates the effect of MDA-protein or pentosidine-protein on intracellular ROS production. Cryopreserved HMVECads were purchased from Life Technologies and cultured according to the company's instructions in Medium 131 with attachment factor (M-131-500) and addition of Microvascular Growth Supplement until confluence, and then left serum-free overnight.

First, HMVECads were treated with 100 μg/ml of one of three solutions. The first solution (MDA-protein) was rat serum albumin (RSA) treated with an equimolar 1 mmol/L solution of malondialdehyde (MDA) and acetaldehyde for 3 days at 37° C. The second solution was pentosidine-RSA, prepared by incubating 0.5 mg of normal RSA with 0.1 M D-glucose in 0.5 ml of 0.1 M sodium phosphate at 37° C. for 14 days. The third solution (the control) was untreated RSA from the same batch. The HMVECads were treated for 15, 30, 60, or 120 minutes. Following the indicated time, intracellular ROS production was determined by a fluorometric analysis.

The results are shown in FIG. 19. “Con” is the control. “MD” is the cells treated with the first solution of MDA-RSA. “Pent” is the cells treated with the second solution of pentosidine-RSA. The results indicated that cells exposed to the solutions for longer times had greater ROS production, as expected inasmuch as SCARB1 is involved with scavenging modified proteins and this scavenging leads to cellular stress.

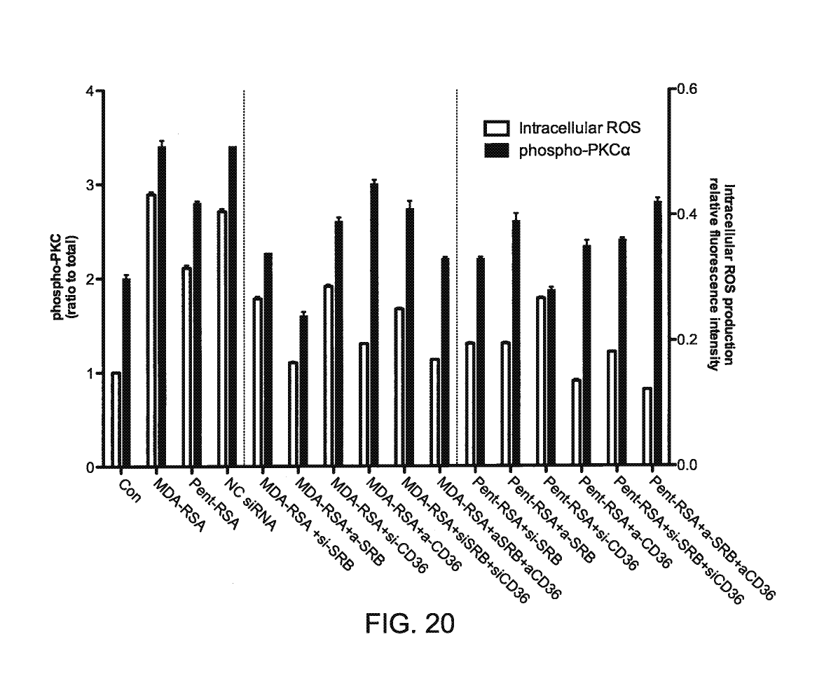

Next, the cells were pre-incubated with a polyclonal anti-SCARB1 antibody (a-SRB) or a polyclonal anti-CD36 antibody (a-CD36) or transfected with either SCARB1 siRNA (si-SRB) or CD36 siRNA (siCD36) before exposure to MDA-RSA or Pent-RSA as above. The pre-incubations were performed in various combinations. The results are shown in FIG. 20. The labels beneath each set of bars indicate which pre-incubation and which exposure was made to that set. The bars labelled “NC-siRNA” were pre-incubated with scramble siRNA and exposed to untreated RSA. The dark bars are read using the left y-axis, and the white bars are read using the right y-axis.

The intracellular ROS was a proxy for NADPH oxidase activation and mitochondrial leakage, both of which are linked to cellular stress and induction of apoptosis. The phospho-PKCα measured membrane-associated PKC signalling induced by oxidation and linked to activation of NADPH oxidase and other cellular stressors.

As expected, addition of MDA-RSA or Pent-RSA caused an increase in activity compared to the control unmodified RSA, i.e. more cellular stress signalling and cellular ROS were produced. In vivo, this is contemplated to result in the modification by ROS of yet more proteins, as well as to stress responses in adjacent cells. The addition of siRNA and antibodies reduced the amount of ROS and cellular stress produced, though not down to the level of the control. There was no significant difference between targeting SCARB1 or CD36. The siRNA did not reduce activity as much as the antibodies; this was expected as antibodies are generally better in this regard. A nonsense siRNA sequence (NC-siRNA) did not have the same effect. There was no additional effect in targeting both CD36 and SCARB1 versus targeting only one. It is hypothesized that other scavenger receptor systems are also involved, so completely silencing the response is not possible.

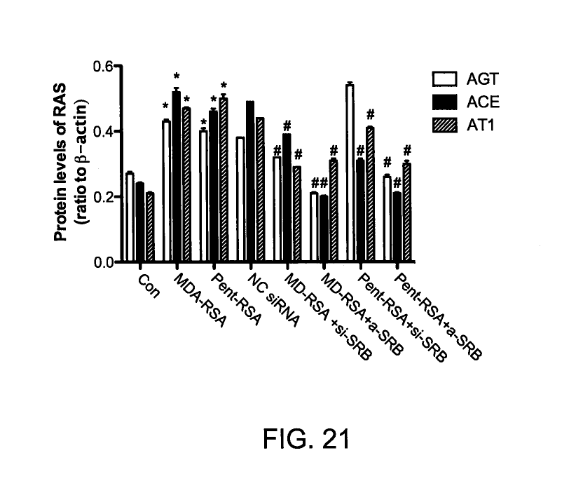

After incubating for 60 minutes, the intracellular ROS production was again determined for the cells of FIG. 20 by a fluorometric analysis. The expression levels of AGT, ACE and AT1 were examined by Western blotting. These results are shown in FIG. 21. These showed the same results: addition of MDA-RSA or Pent-RSA caused an increase in activity compared to the control; and the addition of siRNA and antibodies reduced the amount of modified proteins produced. A nonsense siRNA sequence NC-siRNA did not have the same effect.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A process for ameliorating the effects of oxidatively modified proteins bound to CD36 or SCARB1 by administering a RAS signaling pathway modulator, an oxygen scavenger, an NADPH oxidase inhibitor, a pan-PKC inhibitor, or a PKCα inhibitor to a patient.

2. The process of claim 1, wherein the RAS signaling pathway modulator is enalapril, losartan, or spironolactone.

3. The process of claim 1, wherein the oxygen scavenger is copper-zinc superoxide dismutase, a pterin, a flavonoid, 2,3-dimethyl-6(2-dimethylaminoethyl)-6H-indolo-(2,3-b)quinoxaline, N-acetylcysteine, or ascorbic acid.

4. The process of claim 1, wherein the NADPH oxidase inhibitor is diphenyleneiodonium (DPI), apocynin, a procyanidin, Schisandrin B, or annexin peptide Ac2-26.

5. The process of claim 1, wherein the pan-PKC inhibitor is Gö6983, genistein, calphostin C, or GF109203X.

6. The process of claim 1, wherein the PKCα inhibitor is Gö6976, PKCα (C2-4) inhibitor peptide, aprinocarsen, or MT477.

7. A process for treating complications of renal failure, comprising contacting a biological fluid with a filter body, the filter body having immobilized thereon either a CD36 receptor, a CD36 receptor fragment, a SCARB1 receptor, a SCARB1 receptor fragment, an antibody against a CD36-binding motif, or an antibody against a SCARB1-binding motif, the biological fluid having a reduced quantity of oxidatively modified proteins after contacting the filter body.

8. The process of claim 7, further comprising separating the biological fluid from the filter body.

9. The process of claim 7, wherein the filter body is in the shape of a bead or a hollow fiber.

10. The process of claim 7, wherein the oxidatively modified proteins are advanced oxidation protein products or advanced glycation end products.

11. A process for treating complications of renal failure, comprising administering to a patient an antagonist drug that blocks the binding activity of the RAGE receptor.

12. The process of claim 11, wherein the antagonist drug includes an anti-RAGE antibody, a small interfering RNA configured for silencing RAGE, a synthetic peptide or fusion protein, a modified amino acid, or an antibody targeting RAGE binding motifs or ROS-modifications.

13. The process of claim 11, wherein the antagonist drug is a glitazone, FPS-ZM1, or low anti-coagulant 2-O,3-O-desulfated heparin.

14. A process for treating complications of renal failure, comprising administering to a patient an antagonist drug that blocks the binding activity of the CD36 receptor.

15. The process of claim 14, wherein the antagonist drug includes an anti-CD36 antibody, a small interfering RNA configured for silencing CD36, a synthetic peptide or fusion protein, a modified amino acid, or an antibody targeting CD36 binding motifs or ROS-modifications.

16. The process of claim 14, wherein the antagonist drug is AP5055, AP5258, ursolic acid or a derivative thereof, or hexarelin.

17. A process for treating complications of renal failure, comprising administering to a patient a drug that blocks the binding activity of the SCARB1 receptor, reduces the number of receptors available for binding, or prevents internalization of SCARB1 ligands after binding.

18. The process of claim 17, wherein the drug includes an anti-SCARB1 antibody, a small interfering RNA configured for silencing SCARB1, a synthetic peptide or fusion protein, a modified amino acid, or an antibody targeting SCARB1 binding motifs or ROS-modifications.

19. The process of claim 17, wherein the drug is acetyl-salicylate, sodium-salicylate, ezetimibe, ITX-5061, ITX-7650, or enfuvirtide.

20. A process for controlling the binding of oxidatively modified proteins to SCARB1, comprising administering to a patient a compound that binds to SCARB1 but does not interfere with normal lipoprotein binding.

21. The process of claim 20, wherein the compound is a synthetic peptide or fusion protein, a modified amino acid, an antibody configured against one or more extracellular domains of SCARB1, an antibody targeting SCARB1 binding motifs or ROS-modifications, or an antibody configured for partially blocking SCARB1.

22. A process for identifying patients at risk of tissue amyloid due to reduced renal protein clearance, comprising: measuring the concentration of at least one SCARB1 binding molecule in a bodily fluid of a patient.

23. The process of claim 22, wherein the SCARB1 binding molecule is malondialdehyde (MDA), pentosidine, a protein-malondialdehyde conjugate, or a protein-pentosidine conjugate.

24. A process for identifying patients at risk of complications due to renal failure, comprising: measuring tissue amyloid content, tissue RNA levels of CD36 or SCARB1, or tissue protein levels of CD36 or SCARB1.

25. The process of claim 24, wherein the tissue amyloid content is measured by taking a tissue sample, mixing the tissue sample with a dye, removing excess dye, measuring light absorbance or reflectance spectra in said tissue sample, and comparing the results to a standard curve.

26. The process of claim 24, wherein the tissue amyloid content is measured by introducing a contrast agent to the patient; and subsequently measuring uptake of said contrast agent in tissues.

27. A process for reducing the formation of tissue amyloid due to reduced renal protein clearance, comprising administering to a patient a compound that reduces the cellular effects of modified proteins binding to SCARB1 or CD36, or that blocks said binding.

28. The process of claim 27, wherein the compound is an anti-CD36 antibody, an anti-SCARB1 antibody, a small interfering RNA configured for silencing CD36 or SCARB1, a peptide containing a CD36 or SCARB1 binding motif, a peptide derived from the hepatitis C virus (HCV), AP5055, AP5258, ursolic acid or a derivative thereof, hexarelin, acetyl-salicylate, sodium-salicylate, ezetimibe, ITX-5061, ITX-7650, enfuvirtide, a RAS signaling pathway modulator, an oxygen scavenger, an NADPH oxidase inhibitor, a pan-PKC inhibitor, or a PKCα inhibitor.
